(19) United States
(12) Patent Application Publication
(10) Pub. No.: US 2022/0023394 A1
(43) Pub. Date: Jan. 27, 2022

(54) METHODS FOR DESENSITIZATION OF URICASE

(71) Applicant: John Kenneth Botson, Anchorage, AK (US)

(72) Inventor: John Kenneth Botson, Anchorage, AK (US)

(21) Appl. No.: 17/237,774

(22) Filed: Apr. 22, 2021

Related U.S. Application Data
(60) Provisional application No. 63/013,951, filed on Apr. 22, 2020.

(51) Int. Cl. A61K 38/44; A61K 9/00; C12N 9/06

(52) U.S. Cl. CPC A61K 38/44; C12N 9/0048; A61K 9/0019

(57) ABSTRACT

Disclosed are methods for treating gouty arthropathy in a subject that is allergic to a uricase. In some aspect, disclosed herein are methods for desensitizing a subject allergic to a uricase.

This application claims the benefit of U.S. Provisional Application No. 63/013,951, filed on Apr. 22, 2021 which is incorporated herein in its entirety.

### I. BACKGROUND

Uricases are highly effective in treating refractory gouty arthropathy. Unfortunately, the medication is also highly immunogenic, leading to infusion reaction, loss of drug efficacy, and anaphylaxis. What are needed are methods for desensitizing a hypersensitivity action to uricases.

### II. SUMMARY

Disclosed herein are methods for desensitizing a subject allergic to a uricase, such as Pegloticase, wherein the subject has or is suspected of having gouty arthropathy, said method comprising
1) administering solution 1 to the subject at the rate of about 2 ml/hour with the duration of about 15 min;
2) administering solution 1 to the subject at the rate of about 5 ml/hour with the duration of about 15 min;
3) administering solution 1 to the subject at the rate of about 10 ml/hour with the duration of about 15 min;
4) administering solution 1 to the subject at the rate of about 20 ml/hour with the duration of about 15 min;
5) administering solution 2 to the subject at the rate of about 5 ml/hour with the duration of about 15 min;
6) administering solution 2 to the subject at the rate of about 10 ml/hour with the duration of about 15 min;
7) administering solution 2 to the subject at the rate of about 20 ml/hour with the duration of about 15 min;
8) administering solution 2 to the subject at the rate of about 40 ml/hour with the duration of about 15 min;
9) administering solution 3 to the subject at the rate of about 10 ml/hour with the duration of about 15 min;
10) administering solution 3 to the subject at the rate of about 20 ml/hour with the duration of about 15 min;
11) administering solution 3 to the subject at the rate of about 40 ml/hour with the duration of about 15 min; and
12) administering solution 3 to the subject at the rate of about 80 ml/hour with the duration of about 174.375 min.

In some embodiments, solution 1 comprise about 0.016 mg of the uricase at the concentration of about 0.003 mg/ml, solution 2 comprises about 0.160 mg of the uricase at the concentration of about 0.0032 mg/ml, and solution 3 comprises about 7.937 mg of the uricase at the concentration of about 0.0317 mg/ml.

In some embodiments,
solution 1 administered in step 1) comprises about 0.0002 mg of the uricase,
solution 1 administered in step 2) comprises about 0.0004 mg of the uricase,
solution 1 administered in step 3) comprises about 0.0008 mg of the uricase,
solution 1 administered in step 4) comprises about 0.0016 mg of the uricase,
solution 2 administered in step 5) comprises about 0.0040 mg of the uricase,
solution 2 administered in step 6) comprises about 0.0080 mg of the uricase,
solution 2 administered in step 7) comprises about 0.0160 mg of the uricase,
solution 2 administered in step 8) comprises about 0.0320 mg of the uricase,
solution 3 administered in step 9) comprises about 0.0794 mg of the uricase,
solution 3 administered in step 10) comprises about 0.1587 mg of the uricase,
solution 3 administered in step 11) comprises about 0.3175 mg of the uricase; and
solution 3 administered in step 12) comprises about 232.50 mg of the uricase.

In some embodiments, in total about 8 mg of the uricase is administered to the subject.

In some embodiments, the method of any preceding aspect further comprising administering to the subject at least one anti-inflammatory agent, wherein the at least one anti-inflammatory agent comprises diphenhydramine, cetirizine, famotidine, montelukast, acetaminophen, methylprednisolone, methotrexate, folic acid, or prednisone.

In some aspects, disclosed herein are methods of treating gouty arthropathy in a subject, said method comprising
1) administering solution 1 to the subject at the rate of about 2 ml/hour with the duration of about 15 min;
2) administering solution 1 to the subject at the rate of about 5 ml/hour with the duration of about 15 min;
3) administering solution 1 to the subject at the rate of about 10 ml/hour with the duration of about 15 min;
4) administering solution 1 to the subject at the rate of about 20 ml/hour with the duration of about 15 min;
5) administering solution 2 to the subject at the rate of about 5 ml/hour with the duration of about 15 min;
6) administering solution 2 to the subject at the rate of about 10 ml/hour with the duration of about 15 min;
7) administering solution 2 to the subject at the rate of about 20 ml/hour with the duration of about 15 min;
8) administering solution 2 to the subject at the rate of about 40 ml/hour with the duration of about 15 min;
9) administering solution 3 to the subject at the rate of about 10 ml/hour with the duration of about 15 min;
10) administering solution 3 to the subject at the rate of about 20 ml/hour with the duration of about 15 min;
11) administering solution 3 to the subject at the rate of about 40 ml/hour with the duration of about 15 min; and
12) administering solution 3 to the subject at the rate of about 80 ml/hour with the duration of about 174.375 min.
In some embodiments, the subject has been administered with the uricase prior to step 1). In some embodiment, the method further comprises administering one or more doses of the uricase. In some embodiments, the subject is hypersensitive to the uricase.
In some embodiments, the method disclosed herein further comprises administering to the subject at least one anti-inflammatory agent (e.g., diphenhydramine, cetirizine, famotidine, montelukast, acetaminophen, methylprednisolone, methotrexate, folic acid, or prednisone) and/or at least one therapeutic agent for treating gouty arthropathy (e.g., allopurinol or febuxostat).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows photographs of severe urticarial, hypersensitivity reaction to Pegloticase infusion.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Optional” or “optionally” means that the subsequently described event or circumstance may or may not occur, and that the description includes instances where said event or circumstance occurs and instances where it does not.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of gout), during early onset (e.g., upon initial signs and symptoms of gout), or after an established development of gout. Prophylactic administration can occur for several days to years prior to the manifestation of symptoms of gout.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Therapeutic agent” or “agent” refers to any composition that has a beneficial biological effect. Beneficial biological effects include both therapeutic effects, e.g., treatment of a disorder or other undesirable physiological condition (e.g., gout), and prophylactic effects, e.g., prevention of a disorder or other undesirable physiological condition (e.g., gout). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc. In some embodiments, the therapeutic agent used herein is a uricase (e.g., Pegloticase). In some embodiments, the therapeutic agent used herein is an anti-inflammatory agent.

“Therapeutically effective amount” or “therapeutically effective dose” of a composition (e.g. a composition comprising an agent) refers to an amount that is effective to achieve a desired therapeutic result. In some embodiments, a desired therapeutic result is the control of refractory gouty arthropathy. In some embodiments, a desired therapeutic result is the control of hypersensitivity reaction to Pegloticase. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “hypersensitive” or “allergic” is here defined as abnormally susceptible physiologically to a specific agent via IgE-mediated or other immune responses-mediated mechanisms (as an antigen or drug). Such antigen is in the present specification and claims called an allergen. The allergen in the present invention can be a uricase (e.g., Pegloticase).

The terms “desensitize”, “immunological tolerance” or “tolerance” are here defined as to make (a sensitized or hypersensitive individual) insensitive or nonreactive to a sensitizing agent (as an antigen or drug) by a reduction in immunological reactivity of a host towards specific tolerated antigen(s). The allergen in the present invention can be a uricase (e.g., Pegloticase).

The term “gout” or “gouty arthropathy” refer to a form of arthritis. Gout occurs when urate crystals accumulate in the joint, causing the inflammation and intense pain of a gout attack. Urate crystals can form when high levels of uric acid are found in the blood.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method of Treatment

Uricase, such as Pegloticase, is used in the treatment of refractory gouty arthropathy. Though pegylated, the molecule remains highly immunogenic. It is associated with both immediate and delayed hypersensitivity, manifested as infusion reactions, loss of drug efficacy, and anaphylaxis. Implementation of “monitoring protocols” has been used to identify patients that become inadequate responders due to the development of anti-drug antibodies. These patients are at the highest risk of experiencing a severe infusion reaction with repeated pegloticase exposure. Once a patient experiences a severe infusion reaction, current opinion is that these patients should not be treated with pegloticase again.

Desensitization, a procedure by which patients can be tolerized to medications previously inducing hypersensitivity reactions, has become increasingly more common in oncology to chemotherapy and biologics. Rapid drug desensitization allows immediate administration of an agent which previously caused hypersensitivity, by administering the total dose over a series of very gradual dose adjustments. Successful desensitization to carboplatin as well as biologic medications has been shown but not to uricase.

Accordingly, in some aspects, disclosed herein is a method for desensitizing a subject allergic to a uricase, wherein the subject has or is suspected of having gouty arthropathy, said method comprising
1) administering solution 1 to the subject at the rate of about 0.5 to 6 ml/hour with the duration of about 5 to 30 min;
2) administering solution 1 to the subject at the rate of about 2.5 to 15 ml/hour with the duration of about 5 to 30 min;
3) administering solution 1 to the subject at the rate of about 5 to 30 ml/hour with the duration of about 5 to 30 min;
4) administering solution 1 to the subject at the rate of about 10 to 60 ml/hour with the duration of about 5 to 30 min;
5) administering solution 2 to the subject at the rate of about 2.5 to 15 ml/hour with the duration of about 5 to 30 min;
6) administering solution 2 to the subject at the rate of about 5 to 30 ml/hour with the duration of about 5 to 30 min;
7) administering solution 2 to the subject at the rate of about 10 to 60 ml/hour with the duration of about 5 to 30 min;
8) administering solution 2 to the subject at the rate of about 20 to 120 ml/hour with the duration of about 5 to 30 min;
9) administering solution 3 to the subject at the rate of about 5 to 30 ml/hour with the duration of about 5 to 30 min;
10) administering solution 3 to the subject at the rate of about 10 to 60 ml/hour with the duration of about 5 to 30 min;
11) administering solution 3 to the subject at the rate of about 20 to 120 ml/hour with the duration of about 5 to 30 min; and
12) administering solution 3 to the subject at the rate of about 40 to 240 ml/hour with the duration of about 80 to 480 min.

In some aspects, disclosed herein is a method for desensitizing a subject allergic to a uricase, said method comprising
1) administering solution 1 to the subject at the rate of about 2 ml/hour with the duration of about 15 min;
2) administering solution 1 to the subject at the rate of about 5 ml/hour with the duration of about 15 min;
3) administering solution 1 to the subject at the rate of about 10 ml/hour with the duration of about 15 min;
4) administering solution 1 to the subject at the rate of about 20 ml/hour with the duration of about 15 min;
5) administering solution 2 to the subject at the rate of about 5 ml/hour with the duration of about 15 min;
6) administering solution 2 to the subject at the rate of about 10 ml/hour with the duration of about 15 min;
7) administering solution 2 to the subject at the rate of about 20 ml/hour with the duration of about 15 min;
8) administering solution 2 to the subject at the rate of about 40 ml/hour with the duration of about 15 min;
9) administering solution 3 to the subject at the rate of about 10 ml/hour with the duration of about 15 min;
10) administering solution 3 to the subject at the rate of about 20 ml/hour with the duration of about 15 min;
11) administering solution 3 to the subject at the rate of about 40 ml/hour with the duration of about 15 min; and
12) administering solution 3 to the subject at the rate of about 80 ml/hour with the duration of about 174.375 min.

Accordingly, in some embodiments, solution 1 comprises about 0.016 mg of the uricase at the concentration of about 0.003 mg/ml, solution 2 comprises about 0.160 mg of the uricase at the concentration of about 0.0032 mg/ml, and solution 3 comprises about 7.937 mg of the uricase at the concentration of about 0.0317 mg/ml.

In some embodiments,
solution 1 administered in step 1) comprises at least about 0.0002 mg of the uricase,
solution 1 administered in step 2) comprises at least about 0.0004 mg of the uricase,
solution 1 administered in step 3) comprises at least about 0.0008 mg of the uricase,
solution 1 administered in step 4) comprises at least about 0.0016 mg of the uricase,
solution 2 administered in step 5) comprises at least about 0.0040 mg of the uricase,
solution 2 administered in step 6) comprises at least about 0.0080 mg of the uricase,
solution 2 administered in step 7) comprises at least about 0.0160 mg of the uricase,
solution 2 administered in step 8) comprises at least about 0.0320 mg of the uricase,
solution 3 administered in step 9) comprises at least about 0.0794 mg of the uricase,
solution 3 administered in step 10) comprises at least about 0.1587 mg of the uricase,
solution 3 administered in step 11) comprises at least about 0.3175 mg of the uricase; and
solution 3 administered in step 12) comprises at least about 232.50 mg of the uricase.

Accordingly, in some embodiments, in total at least about 7 mg, 8 mg, 9 mg, 10 mg, 11 mg, 12 mg, 13 mg, 14 mg, 15 mg, 16 mg, 17 mg, 18 mg, 19 mg, 20 mg, 22 mg, 24 mg, 26 mg, 28 mg, 30 mg, 40 mg, 50 mg, 60 mg, or 80 mg of the uricase is administered to the subject. In some embodiments, in total about 8 mg of the uricase is administered to the subject.

It should be understood and herein contemplated that “urate oxidase” or “uricase” is an enzyme that catalyzes the oxidation of highly insoluble uric acid to 5-hydroxyisourate. The uricase disclosed in the invention can be used for treating gouty arthropathy. In some embodiments, the uricase of any preceding comprises Pegloticase. Pegloticase is a recombinant porcine-like uricase drug indicated for the treatment of severe, treatment-refractory, chronic gout. It is a tetrameric protein composed of four identical chains of about 300 amino acid each. Sequence of one chain can be

(SEQ ID NO: 1)
TYKKNDEVEFVRTGYGKDMIKVLHIQRDGKYHSIKEVATTVQLTLSSKKD
YLHGDNSVIPTDTIKNTVNVLAKFKGIKSIETFAVTICEHFLSSFKHVIR
QVYVEEVPWKRFEKNGVKHVHAFIYTPTGTHFCEVEQIRNAGPPVIHSGI
KDLKVLKTTQSGFEGFIKDQFTTLPEVKDRCFATQVYCKWRYHQGRDVDF
EATWDTVRSIVLQKFAGPYDKGEYSPSVQKTLYDIQVLTLGQVPEIEDME
ISIPNIHYLNIDMSKMGLINKEEVLLPLDNPYGKITGTVKRKLSSRL.

In some embodiments, the term “uricase” used herein also refers to any variants, derivatives, or prodrugs of uricase, including, for example, Rasburicase or poly(ethylene glycol) (PEG) conjugate of recombinant porcine-like uricase (PEG-uricase).

In some embodiments, the method of any preceding aspect further comprises administering to the subject at least one anti-inflammatory agent, wherein the at least one anti-inflammatory agent comprises diphenhydramine, cetirizine, famotidine, montelukast, acetaminophen, methylprednisolone, methotrexate, folic acid, or prednisone. The one or more anti-inflammatory agents can be employed to the subject prior to or after the administration of the uricase, or in between the steps of the administration of the uricase.

In some aspects, disclosed herein is a method treating gouty arthropathy in a subject, wherein the subject is allergic to a uricase, said method comprising
1) administering solution 1 to the subject at the rate of about 0.5 to 6 ml/hour with the duration of about 5 to 30 min;
2) administering solution 1 to the subject at the rate of about 2.5 to 15 ml/hour with the duration of about 5 to 30 min;
3) administering solution 1 to the subject at the rate of about 5 to 30 ml/hour with the duration of about 5 to 30 min;
4) administering solution 1 to the subject at the rate of about 10 to 60 ml/hour with the duration of about 5 to 30 min;
5) administering solution 2 to the subject at the rate of about 2.5 to 15 ml/hour with the duration of about 5 to 30 min;
6) administering solution 2 to the subject at the rate of about 5 to 30 ml/hour with the duration of about 5 to 30 min;
7) administering solution 2 to the subject at the rate of about 10 to 60 ml/hour with the duration of about 5 to 30 min;
8) administering solution 2 to the subject at the rate of about 20 to 120 ml/hour with the duration of about 5 to 30 min;
9) administering solution 3 to the subject at the rate of about 5 to 30 ml/hour with the duration of about 5 to 30 min;
10) administering solution 3 to the subject at the rate of about 10 to 60 ml/hour with the duration of about 5 to 30 min;
11) administering solution 3 to the subject at the rate of about 20 to 120 ml/hour with the duration of about 5 to 30 min; and
12) administering solution 3 to the subject at the rate of about 40 to 240 ml/hour with the duration of about 80 to 480 min.

In some aspects, disclosed herein is a method treating gouty arthropathy in a subject, wherein the subject is allergic to a uricase, said method comprising
1) administering solution 1 to the subject at the rate of about 2 ml/hour with the duration of about 15 min;
2) administering solution 1 to the subject at the rate of about 5 ml/hour with the duration of about 15 min;
3) administering solution 1 to the subject at the rate of about 10 ml/hour with the duration of about 15 min;
4) administering solution 1 to the subject at the rate of about 20 ml/hour with the duration of about 15 min;
5) administering solution 2 to the subject at the rate of about 5 ml/hour with the duration of about 15 min;
6) administering solution 2 to the subject at the rate of about 10 ml/hour with the duration of about 15 min;
7) administering solution 2 to the subject at the rate of about 20 ml/hour with the duration of about 15 min;
8) administering solution 2 to the subject at the rate of about 40 ml/hour with the duration of about 15 min;
9) administering solution 3 to the subject at the rate of about 10 ml/hour with the duration of about 15 min;

10) administering solution 3 to the subject at the rate of about 20 ml/hour with the duration of about 15 min;
11) administering solution 3 to the subject at the rate of about 40 ml/hour with the duration of about 15 min; and
12) administering solution 3 to the subject at the rate of about 80 ml/hour with the duration of about 174.375 min.

In some embodiments, the method of any preceding aspect further comprises administering to the subject at least one anti-inflammatory agent comprising diphenhydramine, cetirizine, famotidine, montelukast, acetaminophen, methylprednisolone, methotrexate, folic acid, or prednisone. The one or more anti-inflammatory agents can be employed to the subject prior to or after the administration of the uricase, or in between the steps of the administration of the uricase.

In some embodiments, the subject has been administered with the uricase prior to step 1), wherein the subject is hypersensitive to the uricase. In some embodiments, the subject has been administered with other therapeutic agents for treating gout, such as allopurinol or febuxostat, prior to step 1), wherein the subject is resistant or fails to respond to allopurinol and/or febuxostat.

As noted above, the method disclosed herein can desensitize the subject allergic to the uricase and restore the response to other therapeutic agents for treating gout. Accordingly, in some embodiments, the method of any preceding aspect further comprising administering to the subject one or more doses of the uricase, for example, after step 12). In some embodiments, the method of any preceding aspect further comprising administering to the subject at least one therapeutic agent for treating gouty arthropathy, for example, for example, after step 12), wherein the at least one therapeutic agent for treating gouty arthropathy comprises allopurinol or febuxostat.

As the timing of gouty arthropathy can often not be predicted, it should be understood the disclosed methods of treating, preventing, reducing, and/or inhibiting gouty arthropathy can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the gouty arthropathy; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the gouty arthropathy.

Dosing frequency of employing the method disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiment, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

A) Pharmaceutically Acceptable Carriers

The compositions disclosed herein (e.g., Pegloticase) can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

B) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### V. EXAMPLES

### Example 1: Case Presentation

Pegloticase is a porcine derived uricase used in the treatment of refractory gouty arthropathy. Though pegylated, the molecule remains highly immunogenic. It is associated with both immediate and delayed hypersensitivity, manifested as infusion reactions, loss of drug efficacy, and anaphylaxis. Implementation of “monitoring protocols” has been used to identify patients that become inadequate responders due to the development of anti-drug antibodies. These patients are at the highest risk of experiencing a severe infusion reaction with repeated pegloticase exposure. Once a patient experiences a severe infusion reaction, current opinion is that these patients should not be treated with pegloticase again.

Desensitization, a procedure by which patients can be tolerized to medications previously inducing hypersensitivity reactions, has become increasingly more common in oncology to chemotherapy and biologics. Rapid drug desensitization allows immediate administration of an agent which previously caused hypersensitivity, by administering the total dose over a series of very gradual dose adjustments. Successful desensitization to carboplatin as well as biologic medications has been shown. Disclosed herein is a 3-bag, 12-step protocol for pegloticase which can be used in the outpatient setting.

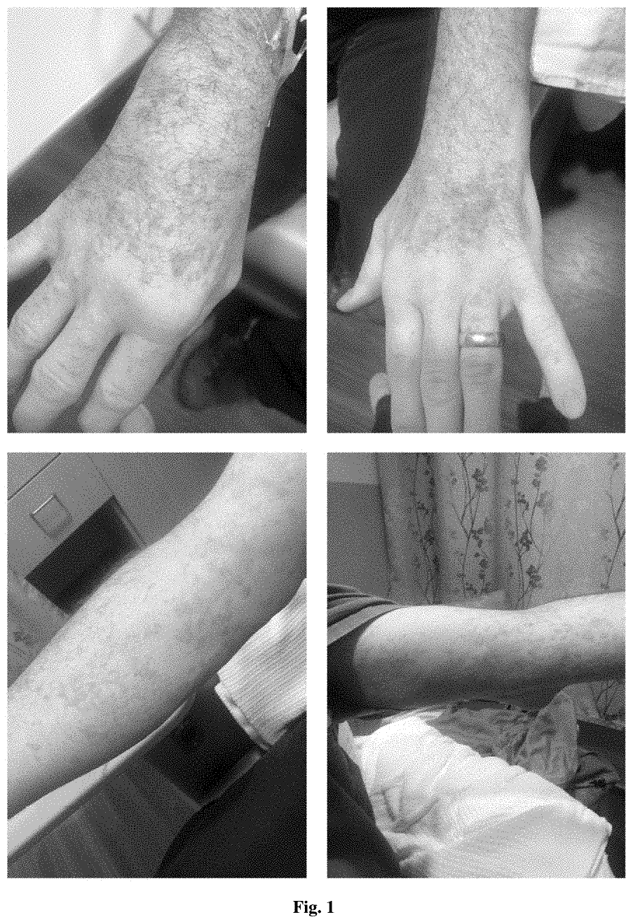

A 48-year-old Caucasian male with >15 years of chronic, refractory, tophaceous, gouty arthropathy who had previously failed allopurinol and febuxostat was undergoing treatment with pegloticase. Standard infusion prophylaxis with oral diphenhydramine 25 mg, oral acetaminophen 975 mg, and IV methylprednisolone 125 mg were given prior to each infusion. The patient was also pretreated with methotrexate 15 mg orally once weekly and folic acid 1 mg orally once daily starting one month prior to initiation of pegloticase to further reduce probability of infusion reactions. During infusion 4, the patient experienced pruritus and urticaria at 41 minutes and the infusion was discontinued. Symptoms resolved with IV diphenhydramine 50 mg. Aggressive pretreatment with oral cetirizine 10 mg and oral prednisone 20 mg the night before the infusion (with standard prophylaxis the day of the infusion) allowed a successful infusion 5. Despite the same regimen, infusion 6 was discontinued at 34 minutes when pruritus and urticaria occurred. Treatment with IV diphenhydramine 50 mg and IV methylprednisolone 50 mg was required. Premedication for infusion 7 included oral prednisone 50 mg and oral cetirizine 10 mg the night before and morning of the infusion. Pruritus and urticaria occurred at 114 minutes. The infusion was held, IV diphenhydramine 25 mg was given, and the infusion was restarted to completion despite persistent urticaria (FIG. 1). At the time of infusion 8, a 3-bag, 12-step rapid desensitization protocol was introduced and the remaining 3 infusions were completed without pruritus, urticaria, or other immediate or delayed hypersensitivity reactions. This represents the first reported case of pegloticase desensitization.

### Example 2: Treatment

Table 1 outlines the pegloticase desensitization protocol. The patient is treated with cetirizine 10 mg orally the night before the infusion. Pretreatment 30 minutes prior to the infusion consists of cetirizine 10 mg, famotidine 20 mg, and montelukast 10 mg orally with methylprednisolone 125 mg IV given over 30 minutes. Each solution is administered successively, changing the rate every 15 minutes per protocol with the patient monitored closely for signs of reactions.

TABLE 1
Example of a 3-bag, 12-step desensitization protocol for pegloticase

| Name of medication: | Pegloticase |
|---|---|
| Target Dose (mg) | 8 |
| Standard volume per bag (ml) | 250 |
| Final rate of infusion (ml/hr) | 80 |
| Calculated target concentration (mg/ml) | 0.032 |
| Standard time of infusion (minutes) | 187.5 |

| | | | | | | Amount of bag |
|---|---|---|---|---|---|---|
| | | | | | Total mg per bag | infused (ml) |
| Solution 1 | 50 | ml of | 0.0003 | mg/ml | 0.016 | 9.25 |
| Solution 2 | 50 | ml of | 0.0032 | mg/ml | 0.160 | 18.75 |
| Solution 3 | 250 | ml of | 0.0317 | mg/ml | 7.937 | 250.00 |

| | | | | Volume | Dose | |
|---|---|---|---|---|---|---|
| | | | | infused | administered | Cumulative |
| | | Rate | Time | per step | with this step | dose |
| Step | Solution | (ml/hr) | (min) | (ml) | (mg) | (mg) |
| 1 | 1 | 2.0 | 15 | 0.50 | 0.0002 | 0.0002 |
| 2 | 1 | 5.0 | 15 | 1.25 | 0.0004 | 0.0006 |
| 3 | 1 | 10.0 | 15 | 2.50 | 0.0008 | 0.0014 |
| 4 | 1 | 20.0 | 15 | 5.00 | 0.0016 | 0.0030 |
| 5 | 2 | 5.0 | 15 | 1.25 | 0.0040 | 0.0070 |
| 6 | 2 | 10.0 | 15 | 2.50 | 0.0080 | 0.0150 |
| 7 | 2 | 20.0 | 15 | 5.00 | 0.0160 | 0.0310 |
| 8 | 2 | 40.0 | 15 | 10.00 | 0.0320 | 0.0630 |
| 9 | 3 | 10.0 | 15 | 2.50 | 0.0794 | 0.1423 |
| 10 | 3 | 20.0 | 15 | 5.00 | 0.1587 | 0.3011 |
| 11 | 3 | 40.0 | 15 | 10.00 | 0.3175 | 0.6186 |
| 12 | 3 | 80.0 | 174.375 | 232.50 | 7.3814 | 8.0000 |
| | | Total time (minutes) = | 339.375 | =5.66 hrs | | |

*** PLEASE NOTE ***
The total volume and dose dispensed are more than the final dose given to patient because the initial solutions are not completely infused.

Following the successful treatment with pegloticase, the patient was restarted on febuxostat 80 mg orally once daily. Though this dose was previously unable to control the gouty arthropathy, the patient continues to do well after 3 months of follow up. The resolution of gouty arthropathy has been demonstrated by maintenance of serum uric acid levels at goal, the absence of new tophi, and lack of gouty flares. The patient continues ongoing evaluation quarterly.

This is the first reported case of a patient successfully desensitized after an infusion reaction to pegloticase. This desensitization protocol represents a significant advancement in treatment of refractory gout with pegloticase, which is used in a population of patients that have no other treatment options. Additional evaluation is performed to determine if desensitization improves efficacy of pegloticase or regains clinical response, which can be driven by neutralizing antibodies.

Pegloticase is a highly effective treatment for refractory gouty arthropathy. Unfortunately the medication is also highly immunogenic, leading to infusion reactions, loss of drug efficacy, and anaphylaxis. Desensitization, a procedure to tolerize a patient to a medication previously causing a hypersensitivity reaction, has been used successfully in oncology for chemotherapy treatment. The same principle can be applied to other specialties. Presented is a 48 year old male that experienced multiple, severe infusion reactions to pegloticase administered for gouty arthropathy. A rapid desensitization was performed using an outpatient, 3-bag, 12-step protocol which allowed multiple additional pegloticase infusions to be performed without incident. This is the first reported case of a patient successfully desensitized after an infusion reaction to pegloticase.

1. A method for desensitizing a subject allergic to a uricase, said method comprising
1) administering solution 1 to the subject at the rate of about 2 ml/hour with the duration of about 15 min;
2) administering solution 1 to the subject at the rate of about 5 ml/hour with the duration of about 15 min;
3) administering solution 1 to the subject at the rate of about 10 ml/hour with the duration of about 15 min;
4) administering solution 1 to the subject at the rate of about 20 ml/hour with the duration of about 15 min;
5) administering solution 2 to the subject at the rate of about 5 ml/hour with the duration of about 15 min;
6) administering solution 2 to the subject at the rate of about 10 ml/hour with the duration of about 15 min;
7) administering solution 2 to the subject at the rate of about 20 ml/hour with the duration of about 15 min;
8) administering solution 2 to the subject at the rate of about 40 ml/hour with the duration of about 15 min;
9) administering solution 3 to the subject at the rate of about 10 ml/hour with the duration of about 15 min;
10) administering solution 3 to the subject at the rate of about 20 ml/hour with the duration of about 15 min;
11) administering solution 3 to the subject at the rate of about 40 ml/hour with the duration of about 15 min; and
12) administering solution 3 to the subject at the rate of about 80 ml/hour with the duration of about 174.375 min.

2. The method of claim 1, wherein solution 1 comprises about 0.016 mg of the uricase at the concentration of about 0.003 mg/ml, solution 2 comprises about 0.160 mg of the uricase at the concentration of about 0.0032 mg/ml, and solution 3 comprises about 7.937 mg of the uricase at the concentration of about 0.0317 mg/ml.

3. The method of claim 1, wherein
solution 1 administered in step 1) comprises about 0.0002 mg of the uricase,
solution 1 administered in step 2) comprises about 0.0004 mg of the uricase,
solution 1 administered in step 3) comprises about 0.0008 mg of the uricase,
solution 1 administered in step 4) comprises about 0.0016 mg of the uricase,
solution 2 administered in step 5) comprises about 0.0040 mg of the uricase,
solution 2 administered in step 6) comprises about 0.0080 mg of the uricase,
solution 2 administered in step 7) comprises about 0.0160 mg of the uricase,
solution 2 administered in step 8) comprises about 0.0320 mg of the uricase,
solution 3 administered in step 9) comprises about 0.0794 mg of the uricase,
solution 3 administered in step 10) comprises about 0.1587 mg of the uricase,
solution 3 administered in step 11) comprises about 0.3175 mg of the uricase; and
solution 3 administered in step 12) comprises about 232.50 mg of the uricase.

4. The method of claim 1, wherein in total about 8 mg of the uricase is administered to the subject.

5. (canceled)

6. The method of claim 1, wherein the uricase is pegloticase.

7. The method of claim 1, wherein the uricase is administered intravenously.

8. The method of claim 1, further comprising administering to the subject at least one anti-inflammatory agent.

9. (canceled)

10. The method of claim 1, wherein the subject has or is suspected of having gouty arthropathy.

11. A method for treating gouty arthropathy in a subject, said method comprising
1) administering solution 1 to the subject at the rate of about 2 ml/hour with the duration of about 15 min;
2) administering solution 1 to the subject at the rate of about 5 ml/hour with the duration of about 15 min;
3) administering solution 1 to the subject at the rate of about 10 ml/hour with the duration of about 15 min;
4) administering solution 1 to the subject at the rate of about 20 ml/hour with the duration of about 15 min;
5) administering solution 2 to the subject at the rate of about 5 ml/hour with the duration of about 15 min;
6) administering solution 2 to the subject at the rate of about 10 ml/hour with the duration of about 15 min;
7) administering solution 2 to the subject at the rate of about 20 ml/hour with the duration of about 15 min;
8) administering solution 2 to the subject at the rate of about 40 ml/hour with the duration of about 15 min;
9) administering solution 3 to the subject at the rate of about 10 ml/hour with the duration of about 15 min;
10) administering solution 3 to the subject at the rate of about 20 ml/hour with the duration of about 15 min;
11) administering solution 3 to the subject at the rate of about 40 ml/hour with the duration of about 15 min; and
12) administering solution 3 to the subject at the rate of about 80 ml/hour with the duration of about 174.375 min.

12. The method of claim 11, wherein solution 1 comprises about 0.016 mg of the uricase at the concentration of about 0.003 m/ml, solution 2 comprises about 0.160 mg of the uricase at the concentration of about 0.0032 mg/ml, and solution 3 comprises about 7.937 mg of the uricase at the concentration of about 0.0317 mg/ml.

13. The method of claim 11, wherein
solution 1 administered in step 1) comprises about 0.0002 mg of the uricase,
solution 1 administered in step 2) comprises about 0.0004 mg of the uricase,
solution 1 administered in step 3) comprises about 0.0008 mg of the uricase,
solution 1 administered in step 4) comprises about 0.0016 mg of the uricase,
solution 2 administered in step 5) comprises about 0.0040 mg of the uricase,
solution 2 administered in step 6) comprises about 0.0080 mg of the uricase,
solution 2 administered in step 7) comprises about 0.0160 mg of the uricase,
solution 2 administered in step 8) comprises about 0.0320 mg of the uricase,
solution 3 administered in step 9) comprises about 0.0794 mg of the uricase,
solution 3 administered in step 10) comprises about 0.1587 mg of the uricase,
solution 3 administered in step 11) comprises about 0.3175 mg of the uricase; and
solution 3 administered in step 12) comprises about 232.50 mg of the uricase.

14. The method of claim 11, wherein in total about 8 mg of the uricase is administered to the subject.

15. The method of claim 11, wherein the subject has been administered with the uricase prior to step 1) in claim 11.

16. The method of claim 11, wherein the subject is hypersensitive to the uricase.

17. The method of any one of claim 11, further comprising administering to the subject one or more doses of the uricase.

18. The method of claim 11, wherein the uricase is pegloticase.

19. The method of claim 11, wherein the uricase is administered intravenously.

20. The method of claim 11, further comprising administering to the subject at least one anti-inflammatory agent.

21. (canceled)

22. The method of claim 11, further comprising administering to the subject at least one therapeutic agent for treating gouty arthropathy.

23. The method of claim 22, wherein the at least one therapeutic agent for treating gouty arthropathy comprises allopurinol or febuxostat.


SEQUENCE LISTING (1 sequence)

SEQ ID NO: 1 - protein, 298 aa, Homo sapiens
TYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL